(19) United States
(12) Patent Application Publication
(10) Pub. No.: US 2019/0307601 A1
(43) Pub. Date: Oct. 10, 2019

(54) Systems and Methods for Providing a Cooling Compression Apparatus for Post-Surgery Recovery

(71) Applicant: HittMac, LLC, Brooklyn, NY (US)

(72) Inventors: Erin Hittman, New York, NY (US); Josh Hittman, New York, NY (US); Adrian Slattery, Rocky River, OH (US); Anna Woo, Cleveland Heights, OH (US)

(73) Assignee: HittMac, LLC

(21) Appl. No.: 15/949,739

(22) Filed: Apr. 10, 2018

(51) Int. Cl. A61F 7/00; A41D 13/12; A61F 9/04; A61F 5/03

(52) U.S. Cl. CPC A61F 7/00; A41D 13/1245; A61F 9/04; A61F 2007/0228; A61F 2007/0021; A61F 2007/0004; A61F 2007/0056; A61F 5/03

(57) ABSTRACT

The present invention includes a garment with an attached inflatable bladder that is designed to be adjacent to the patient's breasts during use for post-surgery recovery. The inflatable bladder has an inlet tube and an outlet tube that connects to a pumping apparatus to supply cold fluid to the inflatable bladder for cooling therapy. The inflatable bladder may include a tortuous water passageway through a first breast section, a second breast section, and a sternum section such that the cool water continues to be recycled through the inflatable bladder without sitting and becoming warm. The garment includes a set of adjustable shoulder straps and a set of adjustable chest straps, which enable the patient to adjust the tightness of the breast cooling and compression apparatus without raising her arms above her head. The present invention also includes a face mask with an inflatable bladder that has an inlet and outlet tube that connects to a pumping apparatus to supply cooling therapy. The inflatable bladder of the face mask includes a tortuous water passageway to recycle the cold water.

### TECHNICAL FIELD

The present invention relates generally to a cooling compression apparatus to assist a patient with post-surgery recovery, and more specifically, to a wrap and bladder system designed to assist with the healing process after breast augmentation or facial surgery.

### BACKGROUND OF THE INVENTION

Plastic surgery, and more specifically, breast augmentation and facial surgery are big business in the United States and around the world. Recent numbers suggest that Americans alone spent $8 billion on plastic surgery in 2016, and that this number continues to grow. The advancements in surgeries, implants, medical devices, recovery solutions, and other technologies are growing along with these numbers. One area where the advancements and technology have been lacking relates to the recovery process for plastic surgery patients. These surgeries, including breast augmentation, breast reduction, face lifts, nose reconstructions, and other procedures, are serious medical procedures that require recovery time.

Plastic surgery patients experience severe pain and discomfort after these medical procedures. For this pain, doctors prescribe extreme medications, including opioids, to ease the pain and discomfort. These drugs include morphine, hydrocodone, oxycodone, OxyContin®, Percocet®, Vicodin®, and others that can lead to patient dependencies. Often these post-surgery prescriptions are the beginning of serious addictions. Thus, a medical device or procedure that limits the pain and discomfort from these types of surgeries can assist in limiting a patient's reliance on these types of medications.

Applying cold temperatures and compression to the area of the body affected by the surgery is known to assist in the healing process. Thus, cooling (or ice) and compression may be recommended by the doctor after surgery, but devices that provide these therapies are lacking for plastic surgery patients. Cooling and compression devices for orthopedic surgeries are widely known, but the breast and face have largely been ignored by medical device manufacturers, forcing patients to use ill-fitting ice or gel packs and even cooling devices for other body parts. Cooling and compression bras and face masks that are specifically tailored to assist with the recovery process would be invaluable to these patients. The speedy recovery time may also reduce patients' dependency on medications, which can help prevent addictions from developing.

After breast augmentation or breast reduction surgery, the patient may have difficulty in raising his or her arms above the shoulders. This can make it difficult for the patient to don a sports bra, surgical bra, or gel pack to the affected area. Because current sports bras or surgical bras are not designed to allow for this type of movement, a new cooling and compression technology that could be donned by the patient without lifting his or her arms above the shoulders is needed. Further, traditional sports and surgical are not fully adjustable, which could be designed to allow the patient to increase or reduce the compression applied to the affected area.

With any facial reconstructive surgery, the reduction of swelling, tightening of the skin, and decrease in the appearance of wrinkles are goals of the surgery and recovery. Conventional ice packs or gel packs that are not specially adapted to the contours of the face are ill-equipped to accomplish these results. Thus, a face mask that is designed to match the patient's facial features and provides cooling and compression to the affected area would be an improvement over the current options. Novel technologies that can improve a plastic surgery patient's recovery outcome are needed.

### BRIEF SUMMARY OF THE INVENTION

The present invention comprises a breast cooling and compression apparatus that is intended to be used in the home or health care facility to assist with a patient's recovery after a breast-related surgery. The apparatus may be prescribed by a doctor. The apparatus pumps cool water through an inflatable bladder that covers the patient's chest area (e.g., breasts and sternum) and provides compression to this area. With these capabilities, the present invention (1) aids in pain reduction, (2) enhances recovery of the affected area, (3) reduces healing time, and (4) reduces swelling through compression.

In some embodiments, the present invention includes a garment with an attached inflatable bladder that is designed to be adjacent to the patient's breasts during use. The inflatable bladder has an inlet tube and an outlet tube that connects to a pumping apparatus to supply cold water to the inflatable bladder for cooling therapy. The inflatable bladder may include a tortuous water passageway so that the cool water continues to be recycled through the inflatable bladder without sitting and becoming warm. The garment includes a set of adjustable shoulder straps and a set of adjustable chest straps, which enable the patient to adjust the tightness of the breast cooling and compression apparatus. Therefore, cooling and compression are applied to the patient to improve recovery time.

In some embodiments, the inflatable bladder includes a left breast section, a right breast section, and a sternum section. The left breast section and the right breast section includes numerous dividers and dams to create the tortuous water passageway. The sternum section, which also includes numerous dividers and dams, connects the water passageway from the left breast section to the right breast section. Thus, the cold water from the pumping apparatus would flow from an inlet tube into one breast section, to the sternum section, to the other breast section, and out of the outlet tube. Additional peninsula sections may be connected to the left breast section and the right breast section to account for various patient and breast sizes. Numerous slits and cutouts may be added to the left breast section, right breast section, and sternum section to improve flexibility and further account for various patient and breast sizes.

In some embodiments, the garment of the breast cooling and compression apparatus may resemble a bra with adjustable straps at the shoulder area and at the chest area. These four straps (right shoulder, left shoulder, right chest, left chest) allow the patient to adjust the tightness of the apparatus in the front of the patient, so that assistance from a health care worker or family member is unnecessary. Further, the adjustable shoulder straps should be designed to enable the patient to don or remove the apparatus without raising her arms about her shoulders. Hook and loop fasteners over the shoulders and at the chest area may be used for these adjustable shoulder and chest straps.

The present invention comprises a face mask cooling and compression apparatus that is intended to be used in the home or health care facility to help with a patient's recovery after a face-related surgery and to avoid wrinkles during the healing process. The apparatus may be prescribed by a doctor. The apparatus accomplishes this by pumping cool water that moves through an inflatable bladder covering the patient's face area and providing compression to this area with a face mask. With these capabilities, the present invention (1) aids in pain reduction, (2) enhances recovery of the affected area, (3) reduces healing time, (4) reduces swelling and inflammation through compression, and (5) decreases wrinkles on the face.

In some embodiments, the present invention includes a face mask with an attached inflatable bladder that is designed to be in contact with the patient's face during use. The inflatable bladder has an inlet tube and an outlet tube that connects to a pumping apparatus to supply cold water to the inflatable bladder. The inflatable bladder provides a tortuous water passageway such that the cool water continues to be recycled through the inflatable bladder without sitting and becoming warm. The face mask may include a set of adjustable head straps, which enable the patient to adjust the tightness of the face mask cooling and compression apparatus. Therefore, cooling and compression are applied to the patient to improve recovery time.

In some embodiments, the inflatable bladder includes numerous dividers and dams to create the tortuous water passageway. Thus, the cold water from the pumping apparatus would flow from an inlet tube into one side of the inflatable bladder and out of the other side through an outlet tube. Positioning of the dividers and dams may be adjusted around the nose and eye areas to adapt to the facial features of the patient. Specifically, eye dams or dividers may be configured to surround and mimic the shape of the eyes of the patient and provide a pocket or open space for the patient's eyes. Nose dams or dividers may surround the nose of the patient and provide fluid passageways that mimic the contours of the nose. In some embodiments, the water passageways run north and south to allow the fluid to run smoothly and fit the profile of the patient's head.

In some embodiments, a flexible nose clip is used to hold the face mask cooling and compression apparatus in place. Further, the inlet tube and the outlet tube of the inflatable bladder may be designed to protrude towards the bottom of the apparatus and on opposite sides. Then, the inlet and outlet tubes of the inflatable bladder can be easily connected to the pumping apparatus by the patient and be out of the way during movement or sleep. For both the breast cooling and compression apparatus and the face mask cooling and compression apparatus, warm fluid could also be applied to the inflatable bladder to provide heat to the breast or face area of the user.

### BRIEF DESCRIPTION OF THE DRAWINGS

For a more complete understanding of the present invention, reference is now made to the following descriptions taken in conjunction with the accompanying drawing, in which:

FIG. 1 shows a breast cooling and compression apparatus according to embodiments of the invention;

FIG. 2 shows a breast cooling and compression apparatus according to embodiments of the invention;

FIG. 3 shows an inflatable bladder for a breast cooling and compression apparatus according to embodiments of the invention;

FIG. 4 shows an alternative view of an inflatable bladder for a breast cooling and compression apparatus according to embodiments of the invention;

FIG. 5 shows a breast cooling and compression apparatus according to embodiments of the invention;

FIGS. 6A and 6B show alternative views of a breast cooling and compression apparatus according to embodiments of the invention;

FIG. 7 shows a movement of cool water through an inflatable bladder for a breast cooling and compression apparatus according to embodiments of the invention;

FIG. 8 shows how a breast cooling and compression apparatus can be donned by a patient according to embodiments of the invention;

FIG. 9 shows how a breast cooling and compression apparatus can be donned on a patient by a health care provider according to embodiments of the invention;

FIG. 10 shows a face mask cooling and compression apparatus according to embodiments of the invention;

FIG. 11 shows an alternative view of the face mask cooling and compression apparatus according to embodiments of the invention;

FIG. 12 shows a connection of a water supply to the inflatable bladder and the movement of cool water for a face mask cooling and compression apparatus according to embodiments of the invention;

FIG. 13 shows an inflatable bladder for a face mask cooling and compression apparatus according to embodiments of the invention;

FIG. 14 shows an alternative view of an inflatable bladder for a face mask cooling and compression apparatus according to embodiments of the invention; and

FIG. 15 shows an inflatable bladder for a face mask cooling and compression apparatus according to embodiments of the invention.

### DETAILED DESCRIPTION OF THE INVENTION

As mentioned above, cooling and compression are both recommended during the recovery for any surgery. For plastic surgery on a woman's breasts (i.e., breast augmentation, breast reduction, etc.), doctors have traditionally recommended (1) ice packs or gel packs for cooling treatment and (2) sports bras or surgical bras for compression treatment. However, these disparate solutions are difficult to manage and uncomfortable for the patient. Up to this point, devices designed to accomplish both of these treatments have been unavailable. The ice or gel packs may be sufficient for cold therapy, but lacking for compression therapy, while sports bras only provide compression. Ice or gel packs are also difficult to use for long periods of time. Further, the incision areas for breast surgery can be in many different places, so the cooling and compression apparatus must be flexible and adapted to this part of the female body.

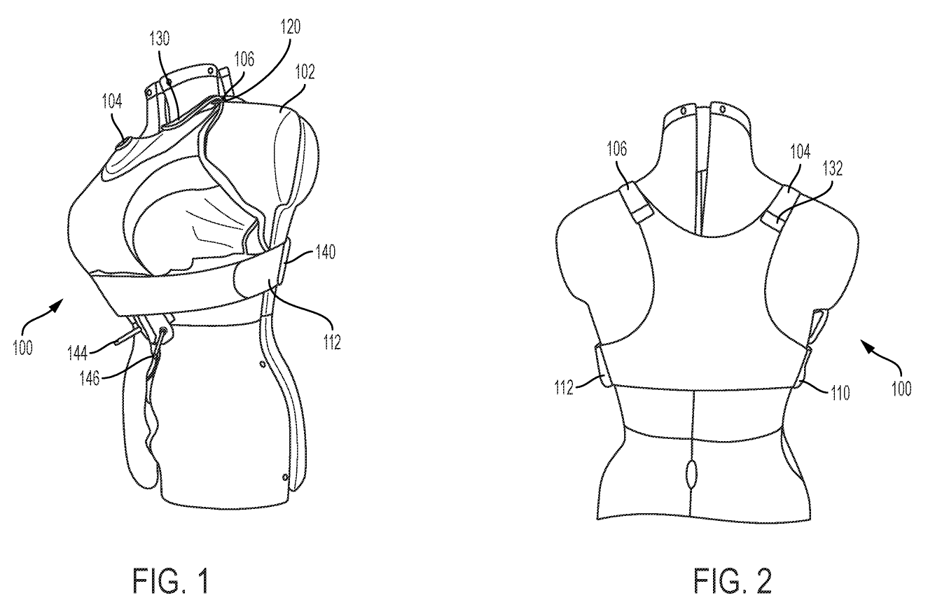

FIG. 1 illustrates a breast cooling and compression apparatus 100, which is designed to be worn by a patient 102 after breast surgery. FIG. 2 illustrates this breast cooling and compression apparatus 100 from an alternative view at the back of the patient 102. The torso shown in FIGS. 1 and 2 represents the patient 102. An inflatable bladder (not shown) inside the breast cooling and compression apparatus 100 assists with the cooling treatment and will be further described below. The breast cooling and compression apparatus 100 resembles a bra with numerous additional features to assist with the compression treatment. While many of these additional features cannot be seen in this view, two adjustable shoulder straps 104, 106 are provided to assist with frontal adjustment. A right adjustable shoulder strap 104 and a left adjustable shoulder strap 106 can be manipulated by the patient 102 to increase or decrease the compression applied to the breast area. These adjustable shoulder straps 104, 106, which are designed to connect over each shoulder of the patient 102, also hold an inflatable bladder (not shown) in place during use. Hook and loop or adhesive mechanisms may be used for attaching and manipulating the adjustable shoulder straps 104, 106. In different embodiments, the adjustable shoulder straps 104, 106 can be adjusted with one strap on each side (right and left) in the front of the patient 102, one strap on each side in the back of the patient 102, or two straps on each side in the front and back of the patient 102. The desired configuration will determine how many hook and loop or adhesive straps and rings or brackets are required for the breast cooling and compression apparatus 100. In an exemplary embodiment, each adjustable shoulder strap 104, 106 has a front shoulder strap 130 and a back shoulder strap 132, which fit into a bracket or ring 120. The bracket or ring 120 holds the front should strap 130 and back shoulder strap 132 in place, while allowing the patient to adjust the fit or compression.

Two adjustable chest straps 110, 112 are provided to assist with chest adjustment and compression. Instead of attaching around the shoulders of the patient 102, these adjustable chest straps 110, 112 attach around a waist or lower chest of the patient 102. A right adjustable chest strap 110 and a left adjustable strap 112 can be manipulated by the patient 102 to increase or decrease the compression applied to the breast area. These adjustable chest straps 110, 112, also assist in holding the inflatable bladder (not shown) in place while attached. Hook and loop or adhesive mechanisms may be used for attaching and manipulating the adjustable chest straps 110, 112. Similar to the adjustable shoulder straps 104, 106, the adjustable chest straps 110, 112 can be adjusted with one strap on each side (right and left) in the front of the patient 102, one strap on each side in the back of the patient 102, or two straps on each side in the front and back of the patient 102. The desired configuration will determine how many hook and loop or adhesive straps and rings or brackets are required for the breast cooling and compression apparatus 100. In an exemplary embodiment, each adjustable chest strap 110, 112 has a single strap that fits into a bracket or ring 140 and connects in the front of the patient 102. The bracket or ring 140 holds the adjustable chest strap 110 in place, while allowing the patient to adjust the fit or compression. The brackets or rings 140 also enable the patient to detach the adjustable shoulder straps or chest straps to assist with donning or removing the apparatus 100.

The adjustable shoulder straps 104, 106 and adjustable chest straps 110, 112 are adjustable in the front of the patient 102 for easy access. This enables the patient to adjust the breast cooling and compression apparatus 100 without assistance from a health care provider, significant other, or family member. The adjustable shoulder straps 104, 106 and adjustable chest straps 110, 112 should be loose and flexible to enable the patient 102 to don and adjust the apparatus 100 by herself. In light of the pain and discomfort from these types of surgeries, these features provide significant advantages over traditional solutions.

The garment portion of the apparatus can be thought of as two panels (dorsal and ventral) that are made of elastic materials. During use, four sets of hook and loop fasteners connect the dorsal panel to the ventral panel (i.e., adjustable shoulder straps 104, 106 and adjustable chest straps 110, 112). In some embodiments, the hook and loop fasteners are designed to be adjusted on the ventral panel for easy access by the patient.

The breast cooling and compression apparatus 100 can be made from various materials, but flexible and temperature resistant materials are used in some embodiments. Flexible materials will assist with providing support and compression for various breast sizes and types of surgeries. And since an inflatable bladder (not shown) will be applying fluids at a cold temperature to the patient, temperature and water-resistant materials will assist with keeping the patient comfortable while using the invention. The design of the apparatus 100 has features to fit a variety of patient body forms and breast shapes. The adjustable shoulder straps 104, 106, adjustable chest straps 110, 112, and the materials provide a one size fits all flexibility that has been unavailable in conventional devices.

FIG. 1 further shows an inlet tube 144 and an outlet tube 146, which are designed to provide fluids to the inflatable bladder (not shown). These tubes 144, 146 and their connection to the inflatable bladder and pumping apparatus (not shown) will be further described below. The inlet tube 144 and outlet tube 146 are exposed and project outwards around a stomach area of the patient for easy access and connection to the pumping apparatus. Cooling fluids enter and circulate through the inflatable bladder at these connections to the inlet tube 144 and outlet tube 146. In certain embodiments, the inlet and outlet tubes 144, 146 may be interchanged or connected at a different area of the breast cooling and compression apparatus 100.

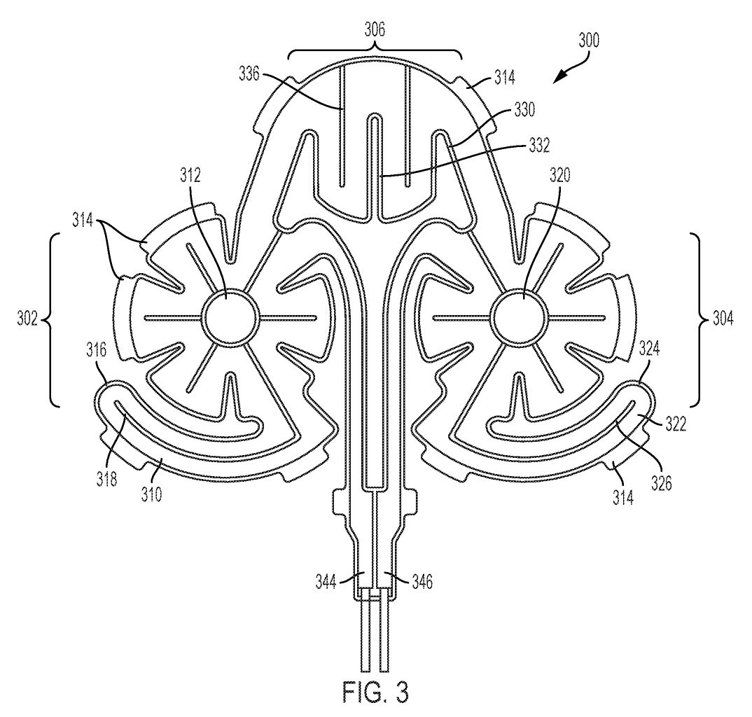

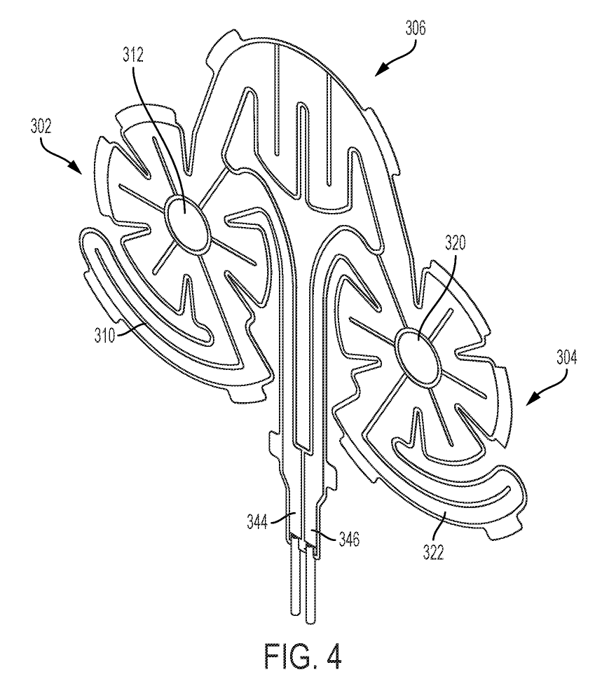

FIG. 3 illustrates an inflatable bladder 300 of a breast cooling and compression apparatus 100. FIG. 4 illustrates an alternative, isometric view of the inflatable bladder 300 of a breast cooling and compression apparatus 100. Throughout the inflatable bladder 300 there are dams or extensions that create a tortuous pathway for the fluid to travel through the inflatable bladder. These features of the present invention are designed to prevent any pockets where water might sit, not move, and become warm. The inflatable bladder 300 may be made from polyvinyl chloride (PVC) or other plastic materials that enable flexible movement, but prevent fluids from escaping the tortuous pathways.

An inlet tube 344 and an outlet tube 346 provide the connection of a fluid pumping apparatus (not shown) to the inflatable bladder 300. Inlet tube 344 and outlet tube 346 correspond to the inlet tube 144 and outlet tube 146 shown in FIG. 1. These tubes 144, 344, 146, 346 could be the same or could be attached through another mechanism. While a pumping apparatus supplies the air or fluid to the inflatable bladder 300, the present invention does not focus on this pumping apparatus, so traditional pumping apparatuses can be used to supply fluids to the inflatable bladder. For example, companies like Breg, Inc. and Stryker Corporation manufacture and sell pumping devices that can connect to the present invention. However, the inlet tubes 144, 344 and outlet tubes 146, 346 are created to be universal adapters to traditional pumping apparatuses. More specifically, the tubes 144, 344, 146, 346 are flexible and similar in size to the connectors or adapters for products that are on the market.

The inflatable bladder 300 consists of three sections areas—a right breast section 302, a left breast section 304, and a sternum section 306. These flexible sections are designed to adapt to different breast sizes and corresponding types of surgery. A right circular section 312 and a right peninsula section 310 make up the right breast section 302. Numerous tabs 314 surround the right circular section 312 and the right peninsula section 310. These tabs 314 are used to be sewn into or attached to the garment or bra-like portion of the breast cooling and compression apparatus 100. Tabs 314 are used to prevent puncturing, damaging, or stretching the inflatable portions of the bladder 300 during assembly or use. In some embodiments, hook and eye connections could be used to connect the inflatable bladder 300 to the apparatus 100. The tortuous pathway for the cool fluid is defined by edges 316 and dams or dividers 318 within the inflatable bladder 300. These edges 316 and dams 318 are sealed so that water cannot leak from the bladder 300 or veer off the tortuous passageway. The right circular section 312 is designed to fit over the patient's breast during use. The right peninsula section 310 is designed to fit over larger breasts, but also fit under smaller breasts. Thus, the flexibility provided by the peninsula section 310 enables the inflatable bladder 300 and cooling technology to cover and conform to multiple patient and breast sizes.

Similar to the right breast section 302, a left circular section 320 and a left peninsula section 322 make up the left breast section 304. Numerous tabs 314 surround the right circular section 320 and the right peninsula section 322. The tortuous pathway for the cool fluid is defined by edges 324 and dams or dividers 326 within the inflatable bladder 300. These edges 324 and dams 326 are sealed so that water cannot leak from the bladder 300 or veer off the tortuous passageway. The left circular section 320 is designed to fit over the patient's breast during use, while the left peninsula section 322 is designed to fit over larger breasts, but also fit under smaller breasts. This configuration provides relief to the various incision sites from breast surgery, including incision sites that are under the patient's breast. The right circular section 312 and the left circular section 320 were designed in circular petal shapes to cover all body and breast sizes. More specifically, the cutouts in the petals (312, 320) enable the inflatable bladder 300 to adjust, reduce, or expand in size to fit different breast sizes.

The sternum section 306 connects the right breast section 302 and the left breast section 304. Numerous tabs 314 surround the sternum section 306 for attachment to the garment or bra-like portion of the breast cooling and compression apparatus 100. The sternum section 306 also includes edges 330 and dams or dividers 336 that create a tortuous pathway for the cool fluid. Not only does the sternum section 306 connect the two breast sections 302, 304, but it cools the patient's sternum, which can be damaged, bruised, sore, and uncomfortable after surgery. Unlike conventional solutions that solely focus on the patient's breasts, the present invention also provides cooling and compression to the patient's sternum to improve recovery outcomes. In addition, the inlet tube 344 and outlet tube 346 run along the patient's sternum to provide added cooling and compression. In some embodiments, the inlet tube 344 and outlet tube 346 are free floating or not attached to each other, except at the distal end where attached to the pumping apparatus (not shown). This separation feature allows the right breast section 302 and left breast section 304 to separate to accommodate different chest circumferences and allows the inlet tube 344 and outlet tube 346 to adjust for the breast height aspect, while still applying cooling therapy to the sternum. A central sternum slot 332 provides additional flexibility for the sternum section 306 by enabling it to adjust and expand for different breast forms, while still allowing the sternum section 306 to rest upon the patient's body during operation.

The sternum section 306 provides increased flexibility and independence for the right breast section 302 and the left breast section 304. The flexible fluid passageways between the sternum section 306 and the breast sections 302, 304, the central sternum slot 332, and the unconnected inlet tube 344 and outlet tube 346, enhance the ability of the right breast section 302 and the left breast section 304 to move independently to fit all body and breast sizes. In some embodiments, the inflatable bladder 300 may include multiple bladders for different portions of the patient's body. For example, the right breast section, left breast section, and sternum section may be three separate bladders that are not connected. In other embodiments, the right breast section and left breast sections are separate, larger sections that also cover the sternum. Further, additional fluid pathways, dams, and dividers for the inflatable bladder 300 are within the scope of the present invention. In some embodiments, the peninsula sections 310, 322 may not be included.

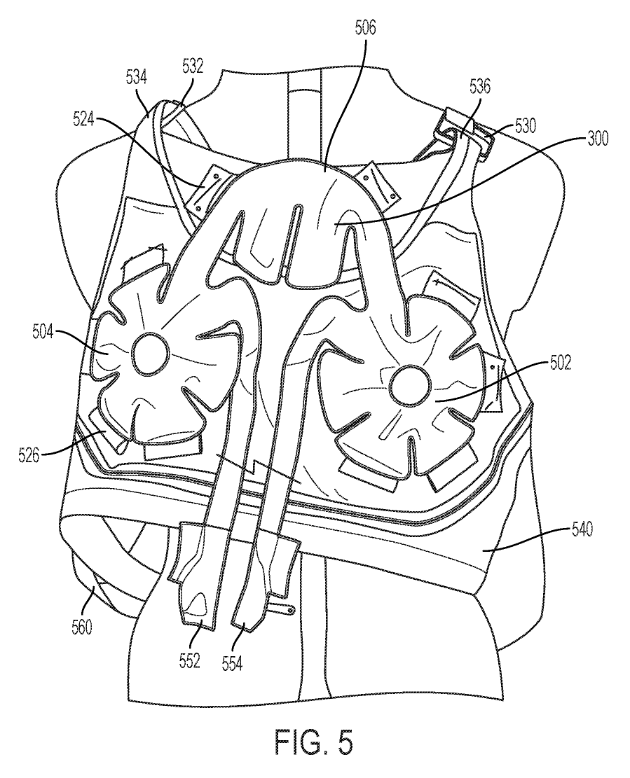

FIG. 5 illustrates a breast cooling and compression apparatus 100 that is partially attached to a patient. More specifically, the bra-like portion of the apparatus 100 is hanging over the back of the patient to expose its interior including an inflatable bladder 300. This inflatable bladder consists of three sections areas—a right breast section 502, a left breast section 504, and a sternum section 506. The peninsula sections shown in FIG. 3 are not shown in this embodiment. Numerous tabs or extensions 524, 526 are used to mount or attach the right breast section 502, left breast section 504, and sternum section 506 to the garment or bra-like portion of the breast cooling and compression apparatus 100. These tabs or extensions 524, 526 can be sewn on, attached with adhesive, connected with a hook and fastener mechanism, or affixed with a connector or fastener. A garment or material surrounding the inflatable bladder 300 that functions as a bra-like portion of the apparatus is shown by reference numeral 540.

An inlet valve or tube 552 and an outlet valve or tube 554 are also shown. These tubes 552, 554 were designed to be easily accessed by the patient for attaching to the pumping apparatus (not shown). Two rings or brackets 530, 532 are used in combination with adjustable shoulder straps 536, 534 to position and hold the apparatus 100 on the patient. To don this breast cooling and compression apparatus 100, the patient must flip the bra-like portion over her head so that the inflatable bladder 300 is adjacent to the patient's breasts. Because the adjustable shoulder straps 534, 536 are already attached, the patient must then attach and manipulate adjustable chest straps 560 to achieve the proper fit. When fully attached, the sternum section 506 would be adjacent to the top of the patient's sternum (right below the neck) and the centers of the right breast section 502 and left breast section 504 would be adjacent to the center or nipple of the patient's breasts.

As for the inflatable bladder 300, it can be designed to touch the patient, to fit in a pocket of the apparatus 100, or be sewn into a material so that the material touches the patient. Materials for the bra-like portion of the apparatus must be selected in view of cooling and condensation concerns.

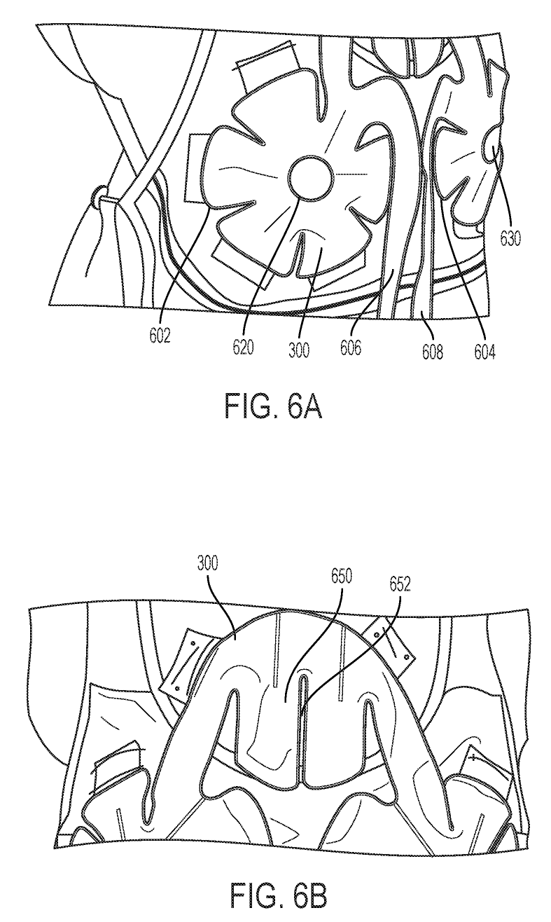

FIGS. 6A and 6B show detailed views of the inflatable bladder 300 of the breast cooling and compression apparatus 100 according to embodiments of the present invention. Numerous cutouts, slits, or voids 602, 604 in the breast sections 620, 630 improve the flexibility of the inflatable bladder 300 and enable it to fit or adapt to most breast sizes. A floating inlet passageway 606 and a floating outlet passageway 608 are designed to be adjacent to the patient's sternum during use to provide cooling treatment. As shown in FIG. 6B, the sternum section 650 also has one or more cutouts, slits, or voids 652 to provide an expandable fit to the patient's sternum area.

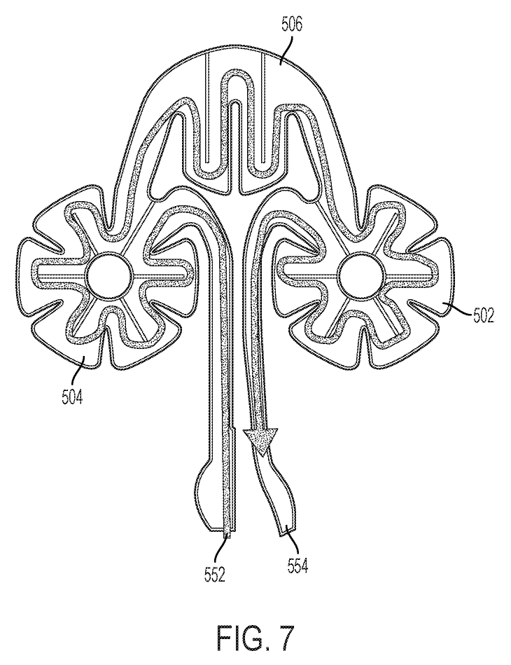

FIG. 7 shows a movement of cold water through an inflatable bladder 300 of a breast cooling and compression apparatus. The cool water from the pumping apparatus (not shown) enters the inflatable bladder at an inlet tube or valve 552. The cool water then travels through the tortuous pathway to the outlet tube or valve 554. Edges and dams within the inflatable bladder 300 create this route that ensures that the cool water continuously travels from inlet valve 552 to outlet valve 554 and avoids any pockets where water might sit, not move, and become warm. The path of the cool water also ensures that the breast areas and the sternum area of the patient receive the cooling benefits of the water. As the fluid fills the inflatable bladder, additional compression is applied to the patient 102. In various embodiments, cool or warm air or fluids may be supplied to the inflatable bladder.

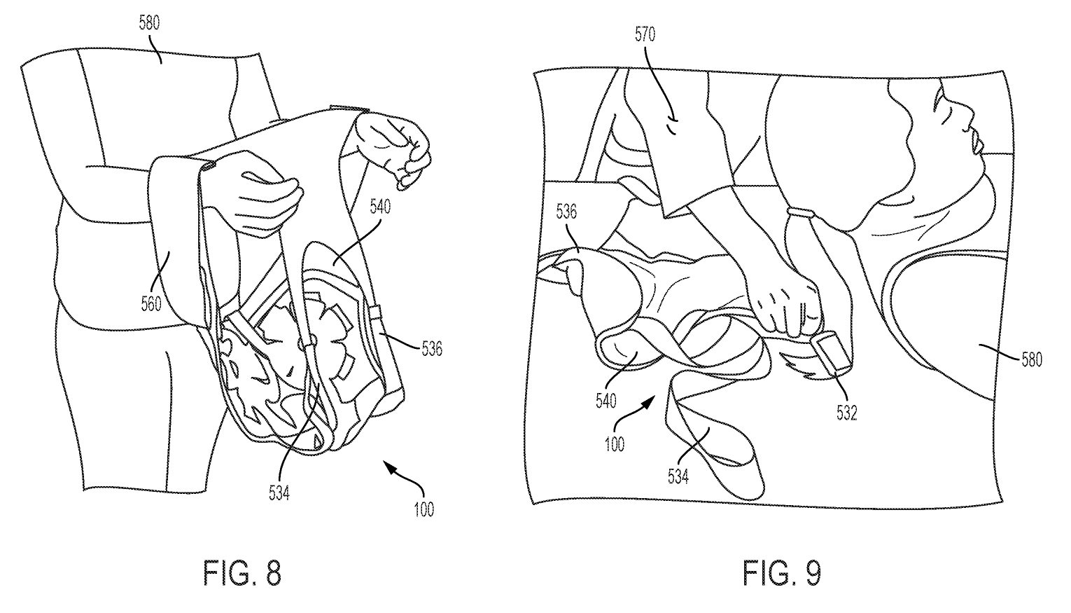

FIG. 8 illustrates how a breast cooling and compression apparatus can be donned by a patient 580, while FIG. 9 illustrates how a breast cooling and compression apparatus can be put on a patient 580 by a health care provider 570 according to embodiments of the invention. Because many plastic surgery patients cannot raise their hands above their shoulders, one of the novel features of the present invention is that the patient can don the breast cooling and compression apparatus 100 without raising her arms above her head. This can be done because of the innovative adjustable shoulder straps and the adjustable chest straps. Initially, the shoulder and chest straps should be in a loose position. As shown in FIG. 8, the patient then uses her arms and hands to slide the bra-portion 540 of the cooling and compression apparatus over her head by lowering her head, allowing the patient to keep her arms below her shoulders while using her hands to get the adjustable shoulder straps 534, 536 over her head. Once the adjustable shoulder straps 534, 536 are over her head and resting on the shoulders, the patient can tighten the adjustable shoulder straps to the desired tightness. Then the patient can tighten the adjustable chest straps 560 to the desired tightness or compression. Hook and loop fasteners for the shoulder and chest straps assist in providing this customized compression.

Because many patients have difficulty moving after a plastic surgery, another novel feature is that a health care provider 570 can don the apparatus on a prone patient 580. As shown in FIG. 9, the health care provider 570 can detach the hook and loop shoulder and chest straps and slide the back of the bra-like portion of the apparatus under the patient 580. When the back of the bra-like portion 540 of the apparatus is fully under the patient 580, the health care provider 570 can pull the front of the bra-like portion 540 of the apparatus over the patient's head, or if the front and back are completely detached, then the health care provider 570 can place the front of the bra-like portion 540 of the apparatus over the patient's sternum and breasts. At this point, the adjustable shoulder straps 534, 536 and the adjustable chest straps can be threaded through or attached to the rings or brackets 532 so that the front and back are now connected. Then the health care provider or patient can tighten or adjust the shoulder straps and chest straps to the desired tightness or compression. Hook and loop shoulder and chest straps assist in providing this customized compression.

After the breast cooling and compression apparatus 100 is fully adjusted, the pumping apparatus (not shown) can be attached. The pumping apparatus includes inlet and outlet tubes or valves that should be connected to the inlet and outlet tubes or valves (FIGS. 5, 7) of the breast cooling and compression apparatus 100. Many different connections or interfaces for the connection of the pumping apparatus (not shown) and the breast cooling and compression apparatus 100 are within the scope of the present invention. After the pumping apparatus is connected, the patient can activate the pumping apparatus to supply the cool fluid to the inflatable bladder. In a preferred embodiment, the pumping apparatus applies cool water to the patient, but cool air or other fluids are within the scope of the present invention.

The patient may wear the breast cooling and compression apparatus 100 from the time period right after surgery until a few weeks after surgery. As with other similar devices, the water temperature should be around 50° F. to provide cooling therapy, but not cause burns or irritation to the patient. Patients can wear the breast cooling and compression apparatus 100 for up to 10-12 hours and can even sleep with it on. As mentioned above, these therapies can reduce recovery time and can limit the patient's need for pain medications, thereby reducing any potential addiction risks from the patient's reliance on these pain medications.

For plastic surgery on a man or woman's face (i.e., rhinoplasty, face lifts, facial balance surgeries, botox, etc.), doctors have traditionally recommended (1) ice packs or gel packs for cooling treatment and (2) face masks or bandages for compression treatment. However, these disparate solutions are difficult to manage and uncomfortable to the patient. Up to this point, devices that are adapted to accomplish both of these treatments have been unavailable. The ice or gel packs may be sufficient for cold therapy, but lacking for compression therapy, while sports bras only provide compression. Ice or gel packs are also difficult to use for long periods of time on an area like the face. Further, the affected areas for facial surgery or procedures can be in many different places, so the cooling and compression apparatus must be flexible and adapted to the face.

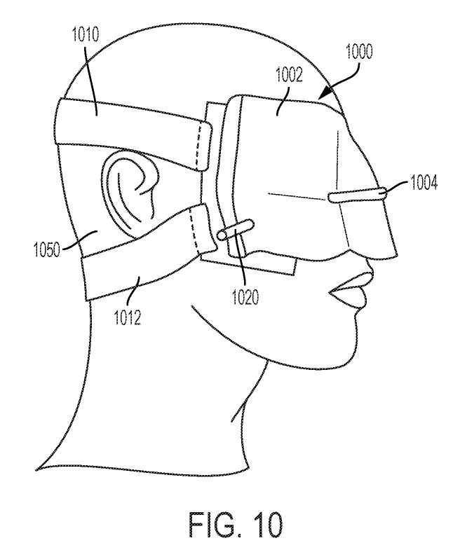

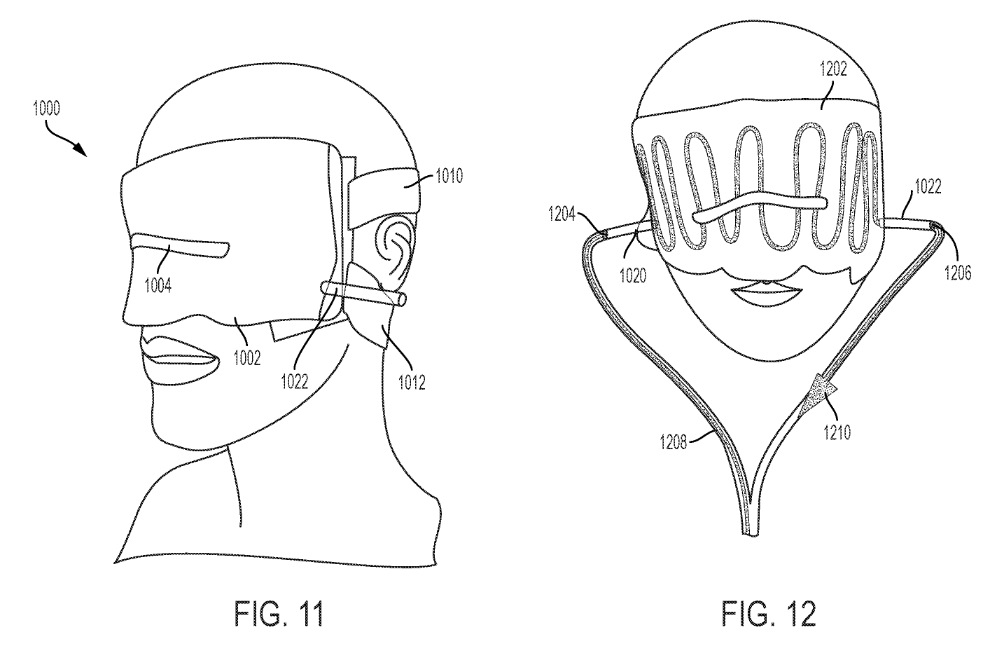

FIG. 10 illustrates a side view of a face mask cooling and compression apparatus 1000, which is designed to be worn by a patient 1050 after plastic surgery. FIG. 11 illustrates a perspective view of this face mask cooling and compression apparatus 1000. An inflatable bladder (not shown) inside the face mask cooling and compression apparatus 1000 assists with the cooling and compression treatment and will be further described below. A face mask 1002 houses the inflatable bladder and is held to the patient's 1050 face by an upper strap 1010 and a lower strap 1012. A nose clip 1004 is used to hold the face mask 1002 in place over the patient's 1050 nose. While the nose clip 1004 is not necessary to hold the face mask 1002 in place, it assists with holding the face mask 1002 in place during sleep or movement of the patient 1050. The nose clip 1004 may be made of deformable aluminum or other materials that are flexible and comfortable to the patient 1050. In other embodiments, adhesive strips, traction ridges, or other clips could be used to hold the face mask 1002 in place. The nose clip 1004 may be attached at or around the nose bridge of the patient 1050. A right tube or valve 1020 is also shown in FIG. 10 and a left tube or valve 1022 is shown in FIG. 11. These tubes or valves 1020, 1022 enable fluid to enter and exit the inflatable bladder (not shown) of the face mask and will be described in further detail below. The tubes or valves 1020, 1022 may be easily accessed and attached to the pumping apparatus (not shown) by the patient 1050.

The nose clip 1004 and straps 1010, 1012 provide flexibility and enable the apparatus 1000 to adapt to various sizes and compression tightness for the patient. The upper strap 1010 and lower strap 1012 may further comprise hook and loop fastener connections at the back of the patient's head to enable the patient to further adjust compression tightness.

FIG. 12 illustrates shows a connection of a fluid supply to an inflatable bladder 1202 according to some embodiments of the present invention. This figure also illustrates the movement of fluid through the inflatable bladder 1202. The outer material of the face mask 1002 is not shown in this figure. The inflatable bladder 1202 can be designed to touch the patient, to fit in a pocket of the face mask 1002 (not shown), or be sewn into a material so that the material touches the patient. Materials for the face mask 1002 of the apparatus 1000 must be selected in view of cooling and condensation concerns. The tubes or valves 1020, 1022 of the inflatable bladder 1202 protrude to provide a connection to the pumping apparatus. Inlet tube 1208 and outlet tube 1210 terminate at corresponding inlet interface 1204 and outlet interface 1206. The inlet interface 1204 connects the tube or valve 1020 to the inlet tube 1208, while the outlet interface 1206 connects the tube or valve 1022 to the outlet tube 1210. Numerous different mechanisms, including insertion, snap fit, valves, can be used to connect the tubes 1208, 1210 and the tubes or valves 1020, 1022. In some embodiments, the inlet and outlet tubes are reversed such that the water flows through the inflatable bladder 1202 in the opposite direction.

Throughout the inflatable bladder 1202 there are dams or extensions that create a tortuous pathway for the fluid to travel through the inflatable bladder. These features of the present invention are designed to prevent any pockets where water might sit, not move, and become warm. The inflatable bladder 1202 may be made from polyvinyl chloride (PVC) or other plastic materials that enable flexible movement but prevent fluids from escaping the tortuous pathways. The cool water from the pumping apparatus (not shown) enters the inflatable bladder at an inlet tube or valve 1020. The cool water then travels through the tortuous pathway to the outlet tube or valve 1022. Edges and dams within the inflatable bladder 1202 create this water passageway that ensures that the cool water continuously travels from inlet valve 1020 to outlet valve 1022 and avoids any pockets where water might sit, not move, and become warm. The path of the cool water also ensures that the face of the patient receive the cooling benefits of the water.

This simple access of the tubing to the pumping apparatus is an improvement over conventional face masks. Specifically, the patient may connect to a pumping apparatus in the same manner that he or she connects to a cell phone or music device. In this configuration, the tubing stays out of the patient's way during relaxation, movement, or sleep.

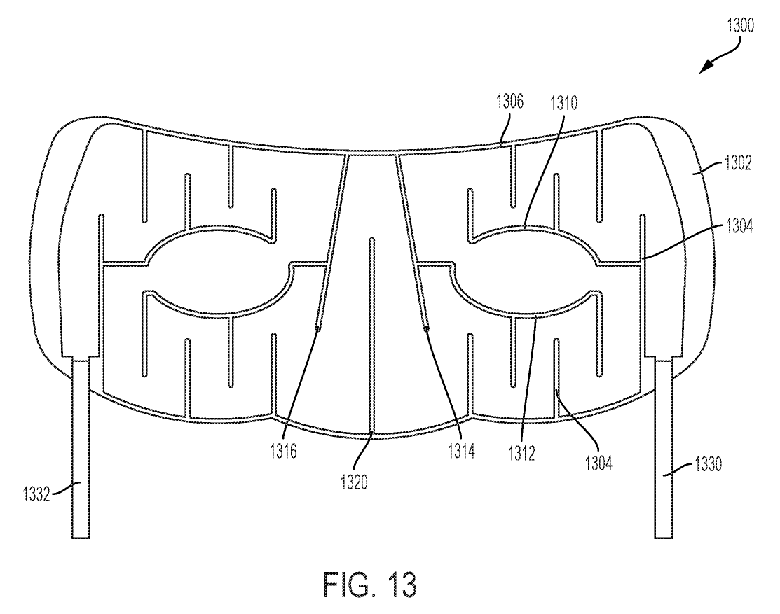

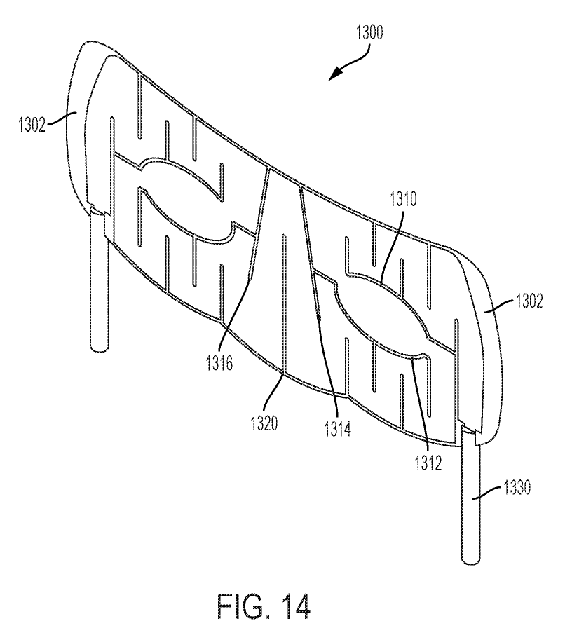

FIG. 13 illustrates an inflatable bladder 1300 for a face mask cooling and compression apparatus 1000 according to embodiments of the invention. FIG. 14 illustrates a perspective view of the inflatable bladder 1300 shown in FIG. 13. An inlet tube or valve 1332 and an outlet tube or valve 1330 supply fluid from the pumping apparatus (not shown) to the inflatable bladder 1300, enabling the fluid to move through the inflatable bladder 1300. Numerous tabs 1302 surround the inflatable bladder 1300. These tabs 1302 are used to be sewn into or attached to outer materials of the face mask cooling and compression apparatus 1000. Tabs 1302 are used to prevent puncturing, damaging, or stretching the inflatable portions of the bladder 1300 during assembly or use. The tortuous pathway for the cool fluid is defined by edges 1306 and dams or dividers 1304 within the inflatable bladder 1300. These edges 1306 and dams 1304 are sealed so that water cannot leak from the bladder 1300 or veer off the tortuous water passageway. As shown in FIG. 14, the inflatable bladder 1300 has a thin profile to provide additional comfort and flexibility to the patient.

In this embodiment, eye and nose dams or dividers are used to provide additional comfort to the patient. Specifically, eye dams or dividers 1310 and 1312 surround the eyes of the patient and provide a pocket or open space for the patient's eyes. These generally elliptical shapes may provide convex surface shapes when inflated with water, which facilitate cooling of the patient's eyes. In some embodiments, these pockets or open spaces for the eyes may be raised to enable movement of the eyelids. These dividers 1310, 1312 are designed to mimic the size and shape of the patient's eyes. Nose dams or dividers 1314, 1316, 1320 surround the nose of the patient and provide fluid passageways that mimic the contours of the nose.

As for the inflatable bladder 1300, it can be designed to touch the patient, to fit in a pocket of the face mask cooling and compression apparatus 1000, or be sewn into a material so that the material touches the patient. Outer materials for the apparatus must be selected in view of cooling and condensation concerns.

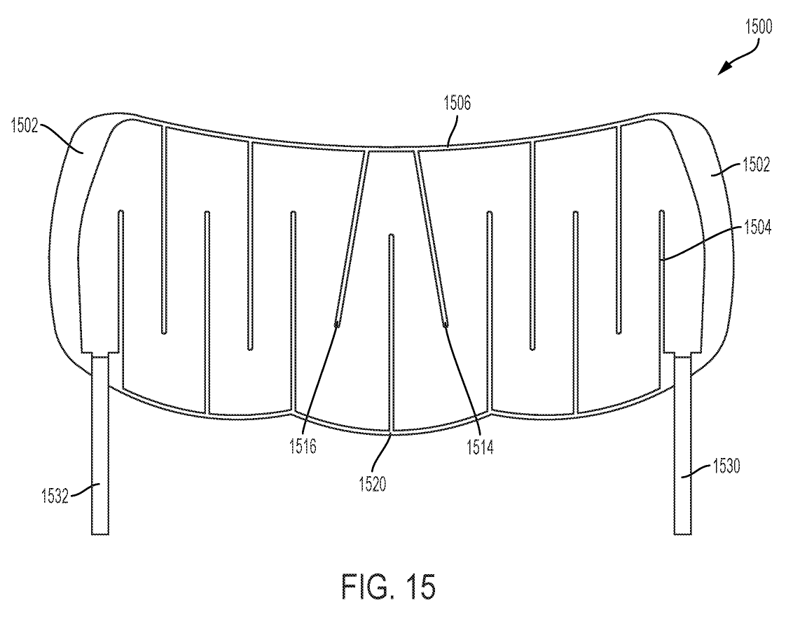

FIG. 15 illustrates an alternative inflatable bladder 1500 for a face mask cooling and compression apparatus 1000 according to embodiments of the invention. An inlet tube or valve 1532 and an outlet tube or valve 1530 supply fluid from the pumping apparatus (not shown) to the inflatable bladder 1500. Numerous tabs 1502, which are used to attach the inflatable bladder 1500 to the outer materials of the face mask cooling apparatus 1000, surround the inflatable bladder 1500. The tortuous pathway for the cool fluid is defined by edges 1506 and dams or dividers 1504 within the inflatable bladder 1500. These edges 1506 and dams 1504 are sealed so that water cannot leak from the bladder 1500 or veer off the tortuous water passageway. Nose dams or dividers 1514, 1516, 1520 surround the nose of the patient and provide fluid passageways that mimic the contours of the nose. In this embodiment, eye dams or dividers are not included.

In some embodiments of the present invention, including FIGS. 13 and 15, the water pathways within the inflatable bladders 1300, 1500 run caudal/cranial or north and south to allow the fluid to run smoothly and fit the profile of the patient's head. These north and south or vertical pathways may provide additional comfort to the patient during operation.

In some embodiments, the inflatable bladder 1300, 1500 may include various configurations. It may include numerous bladders for different portions of the patient's face. For example, the left side and right side of the apparatus may be two separate bladders that are not connected. The portion of the inflatable bladder 1300 that covers the eyes of the patient may be devoid of water. Further, additional fluid pathways, dams, and dividers for the inflatable bladder 1300, 1500 are within the scope of the present invention.

After the face mask cooling and compression apparatus 1000 is fully attached, the pumping apparatus (not shown) can be attached. The pumping apparatus includes inlet and outlet tubes or valves that should be connected to the inlet and outlet tubes or valves (FIGS. 10-12) of the face mask cooling and compression apparatus 1000. Many different connections or interfaces for the connection of the pumping apparatus (not shown) and the face mask cooling and compression apparatus 1000 are within the scope of the present invention. After the pumping apparatus is connected, the patient can activate the pumping apparatus to supply the cool fluid to the inflatable bladder. In a preferred embodiment, the pumping apparatus applies cool water to the patient, but cool air or other fluids are within the scope of the present invention.

The patient may wear the face mask cooling and compression apparatus 1000 from the time period right after surgery until a few weeks after surgery. As with other similar devices, the water temperature should be around 50° F. to provide cooling therapy, but not cause burns or irritation to the patient. Patients can wear the face mask cooling and compression apparatus 1000 for up to 10-12 hours and can sleep with it on. These features of the present invention can improve recovery outcomes and reduce the patient's need for pain medications. While most of this disclosure focused on the application of cold fluid to the patient, warm fluid may also be applied without making substantial changes or improvements to the present invention.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A therapy system, said system comprising:
an inflatable bladder comprising at least a first breast section, a second breast section, and a sternum section, wherein said inflatable bladder is configured to inflate with a fluid; and
a garment that is connected to said inflatable bladder and comprises a least one adjustable shoulder strap, wherein said garment is configured to position said inflatable bladder adjacent to a chest area of a user;
wherein said inflatable bladder is configured to be connected to a fluid supply for supplying said inflatable bladder with said fluid;
wherein said first breast section comprises at least one first set of edges and at least one first set of dividers to create a first fluid passageway;
wherein said sternum section comprises a least one second set of edges and at least one second set of dividers to create a second fluid passageway; and
wherein said second breast section comprises at least one third set of edges and at least one third set of dividers to create a third fluid passageway.

2. The therapy system of claim 1, wherein said first fluid passageway is connected to said second fluid passageway and said second fluid passageway is connected to said third fluid passageway.

3. The therapy system of claim 1, wherein said first set of edges and said first set of dividers create a first tortuous passageway for the fluid to flow through.

4. The therapy system of claim 3, wherein said second set of edges and said second side of dividers create a second tortuous passageway for the fluid to flow through.

5. The therapy system of claim 4, wherein said third set of edges and said third set of dividers create a third tortuous passageway for the fluid to flow through.

6. The therapy system of claim 2, wherein said sternum section provides a flexible connection between said first breast section and said second breast section, wherein said first breast section may move independently of said second breast section.

7. The therapy system of claim 6, wherein said garment comprises a first adjustable shoulder strap and a second adjustable shoulder strap,
wherein said first adjustable shoulder strap comprises a first section and a second section and said second adjustable shoulder strap comprises a third section and fourth section, and
wherein said first section and second section may be secured at a first shoulder of the user and said third section and said fourth section may be secured at a second shoulder of the user to provide compression to the user.

8. The therapy system of claim 7, wherein said user may secure said first section to said second section at the first shoulder and secure said third section to said fourth section at the second shoulder without placing said garment over a head of said user.

9. The therapy system of claim 1, wherein said first breast section and said second breast section are circular shaped with one or more petals surrounding a center of said first breast section and a center of said second breast section.

10. The therapy system of claim 1, wherein said first breast section comprises a first circular portion and a first peninsula portion and said second breast section comprises a second circular portion and a second peninsula portion.

11. The therapy system of claim 1, wherein said inflatable bladder further comprises an inlet tube and an outlet tube for connection to said fluid supply, wherein said inlet tube and said outlet tube are configured to be adjacent to said user's sternum.

12. A therapy apparatus, said apparatus comprising:
an inflatable bladder comprising an inlet tube and an outlet tube for connecting to a fluid supply, wherein said inflatable bladder is configured to inflate with fluid;
a first set of edges in said inflatable bladder to connect said inlet tube to said outlet tube;
a first set of dividers in said inflatable bladder that define a tortuous fluid passageway within said inflatable bladder, wherein said fluid moves through said tortuous fluid passageway from said inlet tube to said outlet tube; and
a wrap that is connected to said inflatable bladder and is configured to be attached to a user to hold said inflatable bladder adjacent to said user;
wherein said first set of dividers are configured to force said fluid to move in a different direction as it passes each of said first set of dividers.

13. The therapy apparatus of claim 12, wherein said wrap comprises a garment with a first shoulder strap and a second shoulder strap for securing said inflatable bladder adjacent to a chest area of said user.

14. The therapy apparatus of claim 13, wherein said inflatable bladder includes a first breast section, a second breast section, and a sternum section.

15. The therapy apparatus of claim 14, wherein said first breast section and said second breast section are circular shaped with one or more petals surrounding a center of said first breast section and a center of said second breast section.

16. The therapy apparatus of claim 14, wherein first breast section comprises a first circular portion and a first peninsula portion and said second breast section comprises a second circular portion and a second peninsula portion.

17. The therapy apparatus of claim 14 wherein said sternum section provides a flexible connection between said first breast section and said second breast section, wherein said first breast section may move independently of said second breast section.

18. A post-surgical breast therapy apparatus, said apparatus comprising:
an inflatable bladder comprising at least an inlet tube, an outlet tube, a first breast section, and a second breast section, wherein said inflatable bladder is configured to inflate with a fluid; and
a garment that is connected to said inflatable bladder and comprises a least two shoulder straps, wherein said garment is configured to position said inflatable bladder adjacent to a chest area of a user;
wherein said inlet tube and said outlet tube are configured to be connected to a fluid supply for supplying said fluid to said inflatable bladder;
wherein said first breast section comprises at least one first set of edges and at least one first set of dividers to create a first tortuous fluid passageway from said inlet tube to said second breast section;
wherein said second breast section comprises at least one second set of edges and at least one second set of dividers to create a second tortuous fluid passageway from said first breast section to said outlet tube.

19. The post-surgical breast therapy apparatus of claim 18, wherein said first set of dividers and said second set of dividers are configured to force said fluid in a different direction as it passes each of said first set of dividers and said second set of dividers.

20. The post-surgical breast therapy apparatus of claim 18, wherein said inflatable bladder further comprises a sternum section that provides a flexible connection between said first breast section and said second breast section.
